(19) United States
(12) Patent Application Publication
(10) Pub. No.: US 2011/0112057 A1
(43) Pub. Date: May 12, 2011

(54) Sequential administration of 20,20,21,21,21-pentafluoro-17-hydroxy-11Beta-[4-(hydroxyacetyl)phenyl]-19-nor-17Alpha-pregna-4,9-dien-3-one and one or more progestogens for the treatment of gynaecological disorders

(75) Inventors: Ulrike Fuhrmann, Berlin (DE); Wolfgang Schwede, Glienicke (DE); Carsten Möller, Berlin (DE)

(73) Assignee: BAYER SCHERING PHARIMA AKTIENGESELL SCHAFT, Berlin (DE)

(21) Appl. No.: 12/992,127

(22) PCT Filed: May 7, 2009
(86) PCT No.: PCT/EP09/03249

§ 371 Date: Jan. 11, 2011

Related U.S. Application Data
(60) Provisional application No. 61/071,725, filed on May 14, 2008.

(30) Foreign Application Priority Data
May 14, 2008 (EP) 08075496.3

(51) Int. Cl. A61K 31/57; A61K 31/585; A61P 15/00

(52) U.S. Cl. 514/170

(57) ABSTRACT

The present invention relates to treatment regimes and combination products of 20,20,21,21,21-pentafluoro-17-hydroxy-11β-[4-(hydroxyacetyl)phenyl]-19-nor-17α-pregna-4,9-dien-3-one of the formula

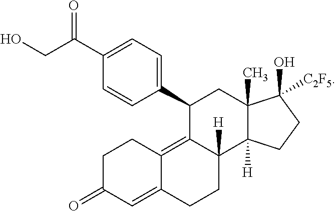

This invention relates in particular to sequential regimes for the treatment of gynaecological disorders in which the abovementioned progesterone antagonist is administered in a first phase, and a progestogen is administered in a second phase.

The present invention relates to treatment regimes and combination products of 20,20,21,21,21-pentafluoro-17-hydroxy-11β-[4-(hydroxyacetyl)phenyl]-19-nor-17α-pregna-4,9-dien-3-one of the formula

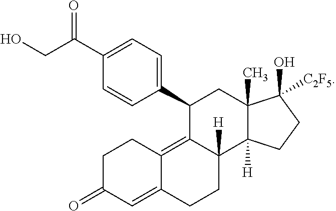

This invention relates in particular to sequential regimes for the treatment of gynaecological disorders in which the abovementioned progesterone antagonist is administered in a first phase, and a progestogen is administered in a second phase.

The invention likewise relates to treatment regimes in which 11β-(4-acetylphenyl)-20,20,21,21,21-pentafluoro-17-hydroxy-19-nor-17α-pregna-4,9-dien-3-one (lonaprisan) is used.

Compounds having antiprogestational activity (competitive progesterone receptor antagonists) were disclosed for the first time in 1982 (RU 486; EP57115) and many have been described since then. Steroids having antiprogestational activity and structurally different from the abovementioned progesterone receptor antagonist, and having a fluorinated 17α side chain, inter alfa also 11β-(4-acetylphenyl)-20,20,21,21,21-pentafluoro-17-hydroxy-19-nor-17α-pregna-4,9-dien-3-one—were published in WO98/34947 and Fuhrmann et al., J. Med, Chem. 43, 5010-5016 (2000).

20, 20, 21, 21, 21-Pentafluoro-17-hydroxy-11β-[4-(hydroxyacetyl)phenyl]-19-nor-17α-pregna-4,9-dien-3-one can be prepared as follows:

i) 11β-(4-Ethenylphenyl)-5-hydroxy-5α-estr-9-ene-3,17-dione 3-(2,2-dimethylpropane-1,3-diyl) ketal

3.3 g of magnesium turnings are introduced under protective gas into 14 ml of absolute tetrahydrofuran, and one drop of 1,2-dibromoethane is added. After onset of the reaction, a solution of 25 g of 4-bromostyrene in 137 ml of absolute tetrahydrofuran is slowly added dropwise in such a way that the internal temperature remains in the range from 40 to 45° C. The reaction mixture is then stirred for one hour until the magnesium has completely reacted. 2.26 g of copper(I) chloride are then added to the mixture. A solution of 8.5 g of 5,10-epoxy-5α,10α-estr-9(11)-ene-3,17-dione 3-(2,2-dimethylpropane-1,3-diyl) ketal (for preparation, see Tetrahedron Lett. 26, 2069-2072 (1985)) in 137 ml of absolute tetrahydrofuran is slowly added dropwise. The reaction mixture is stirred at room temperature for one hour and then poured into saturated aqueous ammonium chloride solution. The aqueous phase is extracted with ethyl acetate, and the organic phases are combined, washed with saturated aqueous sodium chloride solution and dried over sodium sulphate. They are filtered and concentrated in vacuo. Column chromatography on silica gel with a hexane/ethyl acetate mixture affords 6.76 g of the title compound as a colourless foam.

¹H-NMR (300 MHz, CDCl₃): δ=7.30 d (J=9 Hz, 2H, aryl); 7.18 d (J=9 Hz, 2H, aryl); 6.66 dd (J=17 Hz+11 Hz, 1H, vinyl); 5.70 dbr (J=17 Hz, 1H, vinyl); 5.20 dbr (J=11 Hz, 1H, vinyl); 4.44 s (1H, 5-OH); 4.29 dbr (J=6.5 Hz, 1H, H-11); 3.53 m (2H, 3-ketal); 3.51 m (J=11.4 Hz, 1H, 3-ketal); 3.42 m (J=11.4 Hz, 1H, 3-ketal); 1.05 s (3H, 3-ketal); 0.85 s (3H, 3-ketal); 0.49 s (3H, H-18).

ii) 11β-(4-Ethenylphenyl)-20,20,21,21,21-pentafluoro-5,17-dihydroxy-19-nor-5α,17α-pregn-9-en-3-one (2,2-dimethylpropane-1,3-diyl) ketal

A solution of 6.76 g of the compound prepared under i) in 140 ml of absolute toluene is added to 27.9 g of condensed pentafluoroiodoethane in 140 ml of absolute toluene at −78° C. At this temperature, 66.3 ml of a 1.5 molar solution of methyllithium-lithium bromide complex in diethyl ether are added. The mixture is subsequently stirred at 0° C. for one hour. The reaction mixture is then poured into saturated aqueous ammonium chloride solution. Extraction with ethyl acetate is followed by washing with saturated aqueous sodium chloride solution, drying over sodium sulphate and concentrating in vacuo. Chromatography of the resulting crude product on silica gel with a hexane/ethyl acetate mixture affords 3.73 mg of the title compound as a white foam.

¹H-NMR (300 MHz, CDCl₃): δ=7.30 d (J=9 Hz, 2H, aryl); 7.17 d (J=9 Hz, 2H, aryl); 6.67 dd (J=17 Hz+11 Hz, 1H, vinyl); 5.71 dbr (J=17 Hz, 1H, vinyl); 5.20 dbr (J=11 Hz, 1H, vinyl); 4.45 s (1H, 5-OH); 4.31 dbr (J=6.5 Hz, 1H, H-11); 3.53 m (2H, 3-ketal); 3.51 m (J=11.4 Hz, 1H, 3-ketal); 3.42 m (J=11.4 Hz, 1H, 3-ketal); 1.05 s (3H, 3-ketal); 0.85 s (3H, 3-ketal); 0.54 s (3H, H-18).

iii) 11β-[4-(1,2-Dihydroxyethyl)phenyl]-20,20,21,21,21-pentafluoro-5,17-dihydroxy-19-nor-5α,17α-pregn-9-en-3-one (2,2-dimethylpropane-1,3-diyl) ketal

1.68 ml of an aqueous pH 7.00 buffer solution of potassium dihydrogen phosphate and dipotassium hydrogen phosphate, and 206 mg of trimethylamine N-oxide are added to a solution of 1 g of the compound prepared according to ii) in 8.4 ml of tetrahydrofuran. At 0° C., 4.3 ml of a solution of 250 mg of osmium tetroxide in 50 ml of butanol are added dropwise. The reaction mixture is stirred at room temperature for three hours and then poured into saturated aqueous sodium thiosulphate solution. Extraction with ethyl acetate is followed by washing with saturated aqueous sodium chloride solution, drying over sodium sulphate and concentrating in vacuo. Chromatography of the resulting crude product on silica gel with a hexane/ethyl acetate mixture affords 860 mg of the title compound as a white foam. A mixture of epimers at the benzylcarbinol is obtained.

¹H-NMR (300 MHz, CDCl₃): δ=7.25 d (J=9 Hz, 2H, aryl); 7.20 d (J=9 Hz, 2H, aryl); 4.78 m (1H, CHOH); 4.44 s (1H, 5-OH); 4.32 dbr (J=6.5 Hz, 1H, H-11); 3.73 m (1H, CH₂OH); 3.65 m (1H, CH₂OH); 3.54 m (2H, 3-ketal); 3.52 m (J=11.0 Hz, 1H, 3-ketal); 3.44 m (J=11.0 Hz, 1H, 3-ketal); 1.04 s (3H, 3-ketal); 0.87 s (3H, 3-ketal); 0.51 s (3H, H-18).

iv) 11β-[4-(1,2-Dihydroxyethyl)phenyl]-20,20,21,21,21-pentafluoro-17-hydroxy-19-nor-17α-pregna-4,9-dien-3-one

200 mg of the compound described in iii) are stirred in 3 ml of methanol with 141 μl of aqueous half-concentrated sulphuric acid at room temperature for one hour. The mixture is then poured into saturated aqueous sodium bicarbonate solution and extracted with ethyl acetate. The organic phase is washed with saturated aqueous sodium chloride solution, dried over sodium sulphate, filtered and concentrated in vacuo. Column chromatography on silica gel with a hexane/ethyl acetate mixture affords 78 mg of the title compound as a colourless foam. A mixture of epimers at the benzylcarbinol is obtained.

¹H-NMR (300 MHz, CDCl₃): δ=7.25 d (J=9 Hz, 2H, aryl); 7.15 d (J=9 Hz, 2H, aryl); 5.77 s (1H, H-4); 4.74 m (1H, CHOH); 4.42 dbr (J=7 Hz, 1H, H-11); 3.69 m (1H, CH₂OH); 3.59 m (1H, CH₂OH); 0.56 s (3H, H-18).

v) 20,20,21,21,21-Pentafluoro-11β-[4-(hydroxyacetyl)phenyl]-5,17-dihydroxy-19-nor-5α,17α-pregn-9-en-3-one (2,2-dimethylpropane-1,3-diyl) ketal

283 μl of tert-butyl hydroperoxide are added dropwise to 3.6 mg of chromium trioxide in 7 ml of dichloromethane at room temperature. A solution of 450 mg of the compound prepared under iii) in 7 ml of dichloromethane is then added dropwise. The mixture is stirred at room temperature for three hours. The reaction is stopped by adding dimethyl sulphide. The mixture is washed with saturated aqueous sodium chloride solution, dried over sodium sulphate and concentrated in vacuo. Chromatography of the resulting crude product on silica gel with a hexane/ethyl acetate mixture affords 87 mg of the title compound as a white foam.

¹H-NMR (300 MHz, CDCl₃): δ=7.83 d (J=9 Hz, 2H, aryl); 7.37 d (J=9 Hz, 2H, aryl); 4.84 m (2H, CH₂OH); 4.37 dbr (J=6.5 Hz, 1H, H-11); 3.53 m (2H, 3-ketal); 3.47 m (J=11.0 Hz, 1H, 3-ketal); 3.42 m (J=11.0 Hz, 1H, 3-ketal); 1.04 s (3H, 3-ketal); 0.85 s (3H, 3-ketal); 0.49 s (3H, H-18).

vi) 20,20,21,21,21-Pentafluoro-17-hydroxy-11β-[4-(hydroxyacetyl)phenyl]-19-nor-17α-pregna-4,9-dien-3-one

87 mg of the compound described under v) are reacted in analogy to the process described under iv) in 1.4 ml of methanol with 62 μl of aqueous half-concentrated sulphuric acid to give 25 mg of the title compound as a colourless foam.

¹H-NMR (300 MHz, CDCl₃): δ=7.86 d (J=9 Hz, 2H, aryl); 7.34 d (J=9 Hz, 2H, aryl); 5.81 s (1H, H-4); 4.85 dbr (J=5 Hz, 2H, CH₂OH); 4.50 dbr (J=7 Hz, 1H, H-11); 3.50 tbr (J=5 Hz, 1H, OH); 0.57 s (3H, H-18).

Examples 3a and 3b of U.S. Ser. No. 11/984,331 and PCT/EP2007/009997 in which further processes for preparing 20,20,21,21,21-pentafluoro-17-hydroxy-11β-[4-(hydroxyacetyl)phenyl]-19-nor-17α-pregna-4,9-dien-3-one are disclosed are hereby incorporated in the present application.

Administration of substances having an antagonistic property on the progesterone receptor (PR antagonists and SPRMs or selective progesterone receptor modulators) leads—if it takes place over a prolonged period—to changes in the morphology of the endometrium. These changes are important for all long-term uses, whether for the treatment of gynaecological disorders such as endometriosis, of uterine leiomyomas or for the treatment of abnormal or dysfunctional menses. It was possible in preclinical experiments by investigations on primates to demonstrate that PR-antagonistic compounds have an antiproliferative effect on endometrial cells. This is also referred to as non-competitive antioestrogenic effect. This antiproliferative effect was observed to be accompanied by a compaction of the endometrial stroma and a cystic dilatation of the endometrial glands (HODGEN G D, VAN UEM J F, CHILLIK C F, DANFORTH D R, WOLF J P, NEULEN J, WILLIAMS R F, CHWALISZ K: Non-competitive anti-oestrogenic activity of progesterone antagonists in primate models. Hum Reprod (1994) 9 Suppl 1, 77-81; SLAYDEN OD, ZELINSKI-WOOTEN M B, CHWALISZ K, STOUFFER R L, BRENNER R M: Chronic treatment of cycling rhesus monkeys with low doses of the antiprogestin ZK 137 316: morphometric assessment of the uterus and oviduct. Hum Reprod (1998) 13(2), 269-77; SLAYDEN O D, BRENNER R M: RU 486 action after estrogen priming in the endometrium and oviducts of rhesus monkeys (Macaca mulatta). J Clin Endocrinol Metab (1994) 78(2), 440-8). The use of the PR antagonist mifepristone in patients led to similar effects: cystic dilation of the endometrial glands, an inactive endometrial epithelium and a reduced mitotic activity of the endometrium (NARVEKAR N, CAMERON S, CRITCHLEY H O, LIN S, CHENG L, BAIRD D T: Low-dose mifepristone inhibits endometrial proliferation and up-regulates androgen receptor. J Clin Endocrinol Metab (2004) 89(5), 2491-7; BAIRD DT, BROWN A, CRITCHLEY HO, WILLIAMS A R, LIN S, CHENG L: Effect of long-term treatment with low-dose mifepristone on the endometrium. Hum Reprod (2003) 18(1), 61-8). It is additionally known that the use of PR antagonists and SPRMs over a prolonged period—i.e. exceeding the duration of a menstrual cycle—leads to cessation of menstruation. They induce amenorrhoea (NARVEKAR N, CRITCHLEY H O, CHENG L, BAIRD DT: Mifepristone-induced amenorrhoea is associated with an increase in microvessel density and glucocorticoid receptor and a decrease in stromal vascular endothelial growth factor. Hum Reprod (2006) 21(9), 2312-8). The histological changes in the human endometrium induced by administration of PR antagonists and SPRMs cannot be described by conventional pathological categories (HORNE F M, BLITHE D L: Progesterone receptor modulators and the endometrium: changes and consequences. Hum Reprod Update (2007) 13(6): 567-80). Evaluation of such endometrial biopsies is impeded by this unique morphology and by a lack of classification. Thus, for example, an increased thickness of the endometrium was diagnosed in patients receiving mifepristone treatment by means of transvaginal ultrasound. This was possibly caused by the glandular dilation described above. Other studies describe an endometrial hyperplasia without atypia in patients after long-term treatment with mifepristone (EISINGER SH, MELDRUM S, FISCELLA K, et al. Low-dose mifepristone for uterine leiomyomata. Obstet Gynecol 2003; 101:243-50). There is, however, controversy about the assessment of these observations, and re-evaluation of these patients' samples led to a declassification of some patients (Eisinger S H, Bonfiglio T, Fiscella K, et al. Twelve-month safety and efficacy of low-dose mifepristone for uterine myomas. J Minim Invasive Gynecol 2005; 12:227-33). In a recently published study on the effect of the SPRM asoprisnil, a pattern of a “non-physiological secretory effect” was diagnosed in the endometrium after use for 3 months: the endrometrium showed a low level of stromal and glandular proliferation, partially developed secretory glandular properties and stromal changes. In addition, a thickening of the smooth muscle cell layer surrounding the endometrial vessels was described (WILLIAMS A R, CRITCHLEY H O, OSEI J, INGAMELLS S, CAMERON I T, HAN C, CHWALISZ K: The effects of the selective progesterone receptor modulator asoprisnil on the morphology of uterine tissues after 3 months treatment in patients with symptomatic uterine leiomyomata. Hum Reprod (2007) 22(6), 1696-704).

For safe, effective long-term use of PR antagonists in patients, reversibility of the described endometrial effects ought to be ensured, firstly in order to preclude a risk to the safety of the patients, and secondly in order to counter an increased risk of misdiagnosis.

Sequential treatment regimes of competitive progesterone antagonists with progestogens are disclosed for example in U.S. Pat. No. 6,043,234 and of SPRMs with progestogens in US 2005/0215535 and US 2005/0215536.

It is an object of the present invention to provide a medicament which is suitable for safe long-term treatment of gynaecological disorders such as endometriosis, uterine leiomyomas or dysfunctional menses. This medicament is intended, besides efficacy for the indications mentioned, both to ensure a reversibility of the described effects on the endometrium and thus an increased safety of the uterus, make controlled menstruation possible and prevent possible misdiagnoses through the potentially induced thickness of the endometrium.

The present invention achieves this object by the discontinuous use of the progesterone receptor antagonist 20,20,21,21,21-pentafluoro-17-hydroxy-111344-(hydroxyacetyl)phenyl)-19-nor-17α-pregna-4,9-dien-3-one.

For this purpose, the progesterone receptor antagonist 20,20,21,21,21-pentafluoro-17-hydroxy-11β-[4-(hydroxyacetyl)phenyl]-19-nor-17α-pregna-4,9-dien-3-one is administered in one treatment cycle or a plurality of directly consecutive treatment cycles, each treatment cycle consisting of two treatment periods.

In the first treatment period of each treatment cycle, the progesterone receptor antagonist 20,20,21,21,21-pentafluoro-17-hydroxy-11β-[4-(hydroxyacetyl)phenyl]-19-nor-17α-pregna-4,9-dien-3-one is given for 28-168, preferably 56-112, particularly preferably 84, days.

After the end of this first treatment period there follows in each treatment cycle a second treatment period in which no progesterone receptor antagonist is given.

In this second treatment period of each treatment cycle, either progestogen-containing (progestogen-alone products or progestogen combination products for example with oestrogens, such as, for example, commercially available contraceptives or products for hormone replacement therapy) or progestogen-free dose units containing other active ingredients or no dose units at all are administered.

If progestogen-containing dose units are administered in the second treatment period, the duration of this second treatment period is from 1 to 30, preferably from 5 to 20, particularly preferably 7 to 14, days. Second treatment periods with a duration of 7, 10, 11, 12, 14, 21 or 28 days in particular are conceivable.

Suitable progestogens are substances having a contraceptive effect and able to induce withdrawal bleeding. These include for example chlormanidone acetate, cyproterone acetate, desogestrel, dienogest, drospirenone, dydrogesterone, ethynodiol diacetate, etonorgestrel, gestodene, levonorgestrel, medrogestone, medroxyprogesterone and medroxyprogesterone acetate, norethindrone, norethisterone and norethisterone acetate, norgestimate, norgestrel, progesterone, promegestone or trimegestone.

Administration of an oestrogen, for example ethynyloestradiol, oestradiol, oestriol, oestetrol, oestrone or conjugated equine oestrogens would be conceivable as addition, but not necessary. The appropriate progestogen and oestrogen dosages can in part be taken from the US patent application 2005/0215536, paragraph 13-15, which is hereby incorporated into the present application. Further dosage proposals are to be found for example in the 2007 Rote Liste.

Directly thereafter the next treatment cycle would then start with the progesterone receptor antagonists—as described above. The direct administration of the progesterone receptor antagonist following the administration of the progestogen is expected to induce menstruation.

If progestogen-free dose units containing other active ingredients or no dose units at all are administered in the second treatment period, the next treatment cycle—that is renewed administration of the progesterone receptor antagonist in the first treatment period of the following treatment cycle—should be delayed until menstruation occurs (flexible pause). This is expected to take place in most women within 30 days after discontinuation of the progesterone receptor antagonist. However, this menstruation-free period may also be of longer duration in a few cases. A period of 2-4 weeks is preferred in this connection.

It would likewise be conceivable to once again divide the second treatment period of each treatment cycle into two further treatment intervals, with administration of the progestogen-containing dose units, which optionally also have a supplementary oestrogen content, in the first treatment interval, and of the progestogen-free dose units containing other active ingredients or no dose units at all in the second treatment interval which follows directly thereafter. In this case too, the next treatment cycle—that is renewed administration of the progesterone receptor antagonist in the first treatment period of the following treatment cycle—should be delayed until menstruation occurs. This is expected to take place after discontinuation of the progestogen. It likewise appears possible to start the next treatment cycle after the end of the menstruation. Depending on the number of previously administered progestogen-containing daily dose units (e.g. 7, 10, 11, 12, 14, 21), it would also be conceivable to administer n progestogen-free dose units, where n=28−“number of previously administered progestogen-containing daily dose units”.

It is additionally possible to administer further active ingredients—such as, for example, folic acid and salts thereof or metafolin—throughout the treatment or in individual parts thereof, that is to say during the complete treatment cycle, especially during the first and/or second treatment period and/or during the first and/or second treatment interval of the second treatment period. For an illustration of the terms treatment cycle, treatment period and treatment interval, compare the following table:

Treatment cycle

| | Second treatment period | |
|---|---|---|
| | | Second |
| First treatment period | First treatment interval | treatment interval |
| Progesterone receptor | Progestogen administration | Absent |
| antagonist administration | | |
| Progesterone receptor | Progestogen administration | Flexible pause |
| antagonist administration | | |
| Progesterone receptor | Flexible pause | Absent |
| antagonist administration | | |

If the second treatment interval is absent—that is the second treatment period consists only of one, specifically the first, treatment interval—the next treatment cycle follows directly after the end of the progestogen administration or the flexible pause.

Instead of administration of 20,20,21,21,21-pentafluoro-17-hydroxy-11β-[4-(hydroxyacetyl)phenyl]-19-nor-17α-pregna-4,9-dien-3-one, it is conceivable to administer 11β-(4-acetylphenyl)-20,20,21,21,21-pentafluoro-17-hydroxy-19-nor-17α-pregna-4,9-dien-3-one (lonaprisan) with essentially the same clinical result.

The following treatment regimes in particular are conceivable:

| Treatment cycle | | | |
|---|---|---|---|
| | Second treatment period | | |
| First treatment period | 1^{st} treatment interval | 2^{nd} treatment interval | Subsequently |
| 56 days of progesterone receptor antagonist | | Flexible pause | next treatment cycle if app. |
| 56 days of progesterone receptor antagonist | 7 days of progestogen | Flexible pause | next treatment cycle if app. |
| 56 days of progesterone receptor antagonist | 14 days of progestogen | Flexible pause | next treatment cycle if app. |
| 56 days of progesterone receptor antagonist | 21 days of progestogen | Flexible pause | next treatment cycle if app. |
| 56 days of progesterone receptor antagonist | 28 days of progestogen | Flexible pause | next treatment cycle if app. |
| 56 days of progesterone receptor antagonist | 28 days of progestogen/oestrogen | Flexible pause | next treatment cycle if app. |
| 84 days of progesterone receptor antagonist | | Flexible pause | next treatment cycle if app. |
| 84 days of progesterone receptor antagonist | 7 days of progestogen | Flexible pause | next treatment cycle if app. |
| 84 days of progesterone receptor antagonist | 14 days of progestogen | Flexible pause | next treatment cycle if app. |
| 84 days of progesterone receptor antagonist | 21 days of progestogen | Flexible pause | next treatment cycle if app. |
| 84 days of progesterone receptor antagonist | 28 days of progestogen | Flexible pause | next treatment cycle if app. |
| 84 days of progesterone receptor antagonist | 28 days of progestogen/oestrogen | Flexible pause | next treatment cycle if app. |
| 112 days of progesterone receptor antagonist | | Flexible pause | next treatment cycle if app. |
| 112 days of progesterone receptor antagonist | 7 days of progestogen | Flexible pause | next treatment cycle if app. |
| 112 days of progesterone receptor antagonist | 14 days of progestogen | Flexible pause | next treatment cycle if app. |
| 112 days of progesterone receptor antagonist | 21 days of progestogen | Flexible pause | next treatment cycle if app. |
| 112 days of progesterone receptor antagonist | 28 days of progestogen | Flexible pause | next treatment cycle if app. |
| 112 days of progesterone receptor antagonist | 28 days of progestogen/oestrogen | Flexible pause | next treatment cycle if app. |
| 56 days of progesterone receptor antagonist | 7 days of progestogen | Absent | next treatment cycle if app. |
| 56 days of progesterone receptor antagonist | 14 days of progestogen | Absent | next treatment cycle if app. |
| 56 days of progesterone receptor antagonist | 21 days of progestogen | Absent | next treatment cycle if app. |
| 56 days of progesterone receptor antagonist | 28 days of progestogen | Absent | next treatment cycle if app. |
| 56 days of progesterone receptor antagonist | 28 days of progestogen/oestrogen | Absent | next treatment cycle if app. |
| 84 days of progesterone receptor antagonist | 7 days of progestogen | Absent | next treatment cycle if app. |
| 84 days of progesterone receptor antagonist | 14 days of progestogen | Absent | next treatment cycle if app. |
| 84 days of progesterone receptor antagonist | 21 days of progestogen | Absent | next treatment cycle if app. |
| 84 days of progesterone receptor antagonist | 28 days of progestogen | Absent | next treatment cycle if app. |
| 84 days of progesterone receptor antagonist | 28 days of progestogen/oestrogen | Absent | next treatment cycle if app. |
| 112 days of progesterone receptor antagonist | 7 days of progestogen | Absent | next treatment cycle if app. |
| 112 days of progesterone receptor antagonist | 14 days of progestogen | Absent | next treatment cycle if app. |
| 112 days of progesterone receptor antagonist | 21 days of progestogen | Absent | next treatment cycle if app. |
| 112 days of progesterone receptor antagonist | 28 days of progestogen | Absent | next treatment cycle if app. |
| 112 days of progesterone receptor antagonist | 28 days of progestogen/oestrogen | Absent | next treatment cycle if app. |
| 56 days of progesterone receptor antagonist | 7 days of progestogen | 21 days of placebo | next treatment cycle if app. |
| 56 days of progesterone receptor antagonist | 14 days of progestogen | 14 days of placebo | next treatment cycle if app. |
| 56 days of progesterone receptor antagonist | 21 days of progestogen | 7 days of placebo | next treatment cycle if app. |
| 84 days of progesterone receptor antagonist | 7 days of progestogen | 21 days of placebo | next treatment cycle if app. |
| 84 days of progesterone receptor antagonist | 14 days of progestogen | 14 days of placebo | next treatment cycle if app. |
| 84 days of progesterone receptor antagonist | 21 days of progestogen | 7 days of placebo | next treatment cycle if app. |
| 112 days of progesterone receptor antagonist | 7 days of progestogen | 21 days of placebo | next treatment cycle if app. |
| 112 days of progesterone receptor antagonist | 14 days of progestogen | 14 days of placebo | next treatment cycle if app. |
| 112 days of progesterone receptor antagonist | 21 days of progestogen | 7 days of placebo | next treatment cycle if app. |
| 56 days of progesterone receptor antagonist | 21 days of placebo | Absent | next treatment cycle if app. |
| 56 days of progesterone receptor antagonist | 28 days of placebo | Absent | next treatment cycle if app. |
| 84 days of progesterone receptor antagonist | 21 days of placebo | Absent | next treatment cycle if app. |
| 84 days of progesterone receptor antagonist | 28 days of placebo | Absent | next treatment cycle if app. |
| 112 days of progesterone receptor antagonist | 21 days of placebo | Absent | next treatment cycle if app. |
| 112 days of progesterone receptor antagonist | 28 days of placebo | Absent | next treatment cycle if app. |

The progesterone receptor antagonist which is primarily suitable is 20,20,21,21,21-pentafluoro-17-hydroxy-11β-[4-(hydroxyacetyl)phenyl]-19-nor-17α-pregna-4,9-dien-3-one. The progestogens primarily suitable for combination therewith are drospirenone, dienogest or levonorgestrel. Primarily suitable as progestogen/oestrogen combination are commercially available—e.g. in oral contraceptives—combinations. Placebo means not only an active ingredient-free dose form but also dose forms containing active ingredients different from progesterone receptor antagonists, progestogens or progestogen/oestrogen combinations, especially folic acid, salts thereof or metafolin. Instead of the explicitly mentioned periods of the first treatment interval, also suitable at least for blocks 1 to 9 are periods of 10, 11 and 12 days. For reasons of space, express mention of every individual subcombination of specific active ingredients in the regimes disclosed above has been dispensed with. However, it is intended that all the abovementioned combinations in which there is replacement of the words
progesterone receptor antagonist by 20,20,21,21,21-pentafluoro-17-hydroxy-11β-[4-(hydroxyacetyl)phenyl]-19-nor-17α-pregna-4,9-dien-3-one and progestogen by drospirenone;
progesterone receptor antagonist by 20,20,21,21,21-pentafluoro-17-hydroxy-11β-[4-(hydroxyacetyl)phenyl]-19-nor-17α-pregna-4,9-dien-3-one and progestogen by dienogest;
progesterone receptor antagonist by 20,20,21,21,21-pentafluoro-17-hydroxy-11β-[4-(hydroxyacetyl)phenyl]-19-nor-17α-pregna-4,9-dien-3-one and progestogen by levonorgestrel or
progesterone receptor antagonist by 20,20,21,21,21-pentafluoro-17-hydroxy-11β-[4-(hydroxyacetyl)phenyl]-19-nor-17α-pregna-4,9-dien-3-one and progestogen/oestrogen by drospirenone/ethynyloestradiol.

Besides the abovementioned treatment regimes (sequential intake), also suitable—especially for the treatment of endometriosis—is a combination product comprising the progesterone receptor antagonists 20,20,21,21,21-pentafluoro-17-hydroxy-11β-[4-(hydroxyacetyl)phenyl]-19-nor-17α-pregna-4,9-dien-3-one and formulated as fixed combination (simultaneous intake). Instead of formulation as fixed combination, it is also conceivable to formulate the progesterone receptor antagonist 20,20,21,21,21-pentafluoro-17-hydroxy-11β-[4-(hydroxyacetyl)phenyl]-19-nor-17α-pregna-4,9-dien-3-one and the progestogens separately if they are intended for simultaneous intake—especially if they are packaged together.

Regimes in which the words progesterone receptor antagonist are replaced by 11β-(4-acetylphenyl)-20,20,21,21,21-pentafluoro-17-hydroxy-19-nor-17α-pregna-4,9-dien-3-one (lonaprisan) would likewise be conceivable—including the abovementioned replacements of the words progestogen or progestogen/oestrogen.

Oral dose forms to be taken once a day are preferred in all the abovementioned treatment regimes. These can be combined in a medicament package for sequential intake and be combined with a leaflet giving information about the correct sequence of intake.

20,20,21,21,21-Pentafluoro-17-hydroxy-11β-[4-(hydroxyacetyl)phenyl]-19-nor-17α-pregna-4,9-dien-3-one can be formulated by processes familiar to the person skilled in the art using physiologically and pharmacologically acceptable excipients and additives. The same applies to 11β-(4-acetylphenyl)-20,20,21,21,21-pentafluoro-17-hydroxy-19-nor-17α-pregna-4,9-dien-3-one (lonaprisan). Formulations of progestogens and progestogen/oestrogen combinations likewise form part of the state of the art and can be found therein.

The following examples serve to illustrate the invention without restricting it in any way.

### EXAMPLE 1

36 healthy, tube-ligated women from 24 to 43 years of age were subjects in a randomised study on the effect of lonaprisan. For this purpose, the women were divided into three groups: 7 women received placebo, 14 women 1 mg of lonaprisan and 15 women 10 mg of lonaprisan over a period of 3 months, once a day. The continuous treatment by daily administration of lonaprisan led to inhibition of ovulation in the patients and a bleeding pattern which corresponded to amenorrhoea or a greatly reduced bleeding. Neither of these effects were observed in the placebo group. After discontinuation of lonaprisan there was a spontaneous return of menstrual bleeding. In addition, no persistent changes in the endometrium of the patients was found by endometrial biopsies after the first menstrual bleeding after completion of the treatment phase.

### EXAMPLE 2

Women of reproductive age are treated in a randomised study sequentially in two treatment cycles with 20,20,21,21,21-pentafluoro-17-hydroxy-11β-[4-(hydroxyacetyl)phenyl]-19-nor-17α-pregna-4,9-dien-3-one. For this purpose, 20,20,21,21,21-pentafluoro-17-hydroxy-11β-[4-(hydroxyacetyl)phenyl]-19-nor-17α-pregna-4,9-dien-3-one is administered to the women in each case for 84 days with a daily dose of between about 1 mg and about 50 mg, followed by intake of a progestogen over a period of about 10 to 14 days. This complete treatment cycle is repeated once. Clinical end points are the respective pathological symptoms, and the bleeding pattern of the women, the inhibition of ovulation and the endometrial morphology. Sequential treatment of the women with 20,20,21,21,21-pentafluoro-17-hydroxy-11β-[4-(hydroxyacetyl)phenyl]-19-nor-17α-pregna-4,9-dien-3-one and the progestogen leads to a menstrual bleeding following administration of the progestogen. During the phase of treatment with 20,20,21,21,21-pentafluoro-17-hydroxy-11β-[4-(hydroxyacetyl)phenyl]-19-nor-17α-pregna-4,9-dien-3-one, the women show a bleeding pattern which corresponds to amenorrhoea or a greatly reduced bleeding. Endometrial biopsies after completion of each treatment cycle showed no persistent changes in the endometrium of the patients.

1. Pharmaceutical combination product comprising
individual dose units of the progesterone receptor antagonist 20,20,21,21,21-pentafluoro-17-hydroxy-11β-[4-(hydroxyacetyl)phenyl]-19-nor-17α-pregna-4,9-dien-3-one of the formula 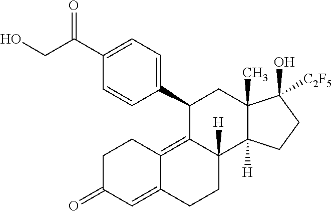
and
individual dosage units of a progestogen
for sequential oral administration thereof for the treatment of gynaecological disorders.

2. Combination product according to claim 1 comprising 28 to 168 dose units of the progesterone receptor antagonist.

3. Combination product according to claim 2 comprising 56 to 112 dose units of the progesterone receptor antagonist.

4. Combination product according to claim 3 comprising 84 dose units of the progesterone receptor antagonist.

5. Combination product according to claim 1 comprising 1 to 30 dose units of the progestogen.

6. Combination product according to claim 5 comprising 5 to 20 dose units of the progestogen.

7. Combination product according to claim 6 comprising 7 to 14 dose units of the progestogen.

8. Combination product according to claim 1 comprising 7, 10, 11, 12, 14, 21 or 28 dose units of the progestogen.

9. Combination product according to claim 1 comprising drospirenone, dienogest or levonorgestrel as progestogen.

10. Combination product according to claim 1 for the treatment of gynaecological disorders, especially uterine leiomyomas, endometriosis or breast cancer.

11. Pharmaceutical product comprising
individual dose units of the progesterone receptor antagonist 20,20,21,21,21-pentafluoro-17-hydroxy-11β-[4-(hydroxyacetyl)phenyl]-19-nor-17α-pregna-4,9-dien-3-one of the formula 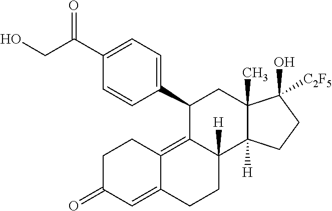
and
information for use from which it is evident that the next treatment cycle is to be started only on or after onset of the menstrual bleeding.
